(19) United States
(12) Patent Application Publication
(10) Pub. No.: US 2025/0213173 A1
(43) Pub. Date: Jul. 3, 2025

(54) DETECTING AND MEASURING FAST OPTICAL SIGNALS (FOS) FROM ONE OR MORE CORNEAL NERVES FOR MONITORING A PATIENT'S HEALTH AND WELLNESS

(71) Applicant: Case Western Reserve University, Cleveland, OH (US)

(72) Inventors: Matthew T. MCPHEETERS, Cleveland, OH (US); Brecken J. BLACKBURN, Cleveland, OH (US); Michael W. JENKINS, Cleveland, OH (US); Andrew M. ROLLINS, Cleveland, OH (US); William J. DUPPS, Cleveland, OH (US)

(21) Appl. No.: 18/851,170

(22) PCT Filed: Mar. 31, 2023
(86) PCT No.: PCT/US2023/017021

§ 371 Date: Sep. 26, 2024

Related U.S. Application Data
(60) Provisional application No. 63/325,653, filed on Mar. 31, 2022.

(51) Int. Cl. A61B 5/00; A61B 3/00; A61B 3/10

(52) U.S. Cl. CPC A61B 5/4041; A61B 3/0025; A61B 3/102

(57) ABSTRACT

An optical method (400, 500) can be performed (402) on a cornea of a patient for a time period to detect transient optical changes in one or more nerves of the cornea corresponding to Fast Optical Signals (FOS). A computing device (102) can receive a record of the FOS of the action potentials generated by the one or more nerves of the cornea and analyze (404) the FOS of the action potentials generated by the one or more nerves of the cornea. The analysis (404) can include quantifying (406) a property of the FOS of the action potentials generated by the one or more nerves of the cornea for the time period and comparing (408) the property of the FOS of the action potentials generated by the one or more nerves of the cornea for the time period to a baseline FOS property. A health property of the cornea can be determined based on the comparison (410).

### CROSS-REFERENCE TO RELATED APPLICATIONS

This application claims the benefit of U.S. Provisional Application No. 63/325,653, filed Mar. 31, 2022, entitled, “MEASURING FAST OPTICAL SIGNALS FROM ONE OR MORE CORNEAL NERVES USING OPTICAL COHERENCE TOMOGRAPHY”. This application is hereby incorporated by reference in its entirety for all purposes.

### GOVERNMENT FUNDING

This invention was made with government support under EY031525, HL134622, and EY028667 awarded by the National Institutes of Health. The government has certain rights in the invention.

### TECHNICAL FIELD

The present disclosure relates generally to optically monitoring a patient's health, and more specifically, to detecting and measuring fast optical signals (FOS) from one or more nerves in the patient's cornea by performing an optical method (e.g., using optical coherence tomography (OCT)) for monitoring the patient's health and wellness.

### BACKGROUND

A patient's cornea is the substantially transparent outer layer of the eye that is highly innervated with sensory nerves that play an important role in stimulating defensive reflexes such as tearing and blinking. Factors related to a patient's overall health can be monitored by studying corneal nerve function. Corneal nerve dysfunction is a significant and growing public health problem that can be correlated to many diseases and conditions. Corneal nerve dysfunction can be caused and/or affected by age, damage, ocular surgeries, and various diseases that cause peripheral neuropathies.

Technological limitations have hampered the assessment of nerve function in corneal nerves of living patients. Nerve function has traditionally been assessed as a single, unidimensional property using aesthesiometers, tonometers, or the like to stimulate the cornea to quantitatively measure sensation changes. However, nerve function in the cornea is highly redundant and measurable loss of sensation with traditional methods may only occur after significant loss of nerve density and function. There is a significant need for methods and systems that can more accurately and precisely characterize corneal nerve function in human patients before significant loss has occurred. Fast optical signals (FOS) are known to correlate with the conduction of nerves, but the FOS have not been used to study corneal nerve function.

### SUMMARY

Fast optical signals (FOS) have been shown to correspond to action potential firing and can be measured in thin, transparent tissues using a high resolution, non-contact optical method (e.g., imaging using optical coherence tomography (OCT)). Corneas are a good target for measuring FOS because corneas are thin, largely transparent, and highly innervated. Accordingly, FOS of corneal nerves can be detected and measured to assess corneal nerve function more accurately and precisely than done previously. In some instances, measuring the FOS of the corneal nerves can lead to earlier detection of corneal conditions such as loss of sensation or other functions. Aspects of the patient's health and wellness can be monitored and assessed via the FOS measurements and the nerve function measurements.

In some instances, a method (e.g., an OCT-based method) can be used for measuring FOS from one or more nerves in the patient's cornea. The method includes performing an optical method on a cornea of a patient for a time period to detect transient optical changes in one or more nerves of the cornea corresponding to FOS provided with generation of action potentials by the one or more nerves of the cornea; receiving, by a computing device comprising a processor, a record of the FOS of the action potentials of the one or more nerves of the cornea; and analyzing the FOS of the action potentials generated by the one or more nerves of the cornea, the analyzing comprising: quantifying a property of the FOS of the action potentials generated by the one or more nerves of the cornea for the time period and comparing the property of the FOS of the action potentials generated by the one or more nerves of the cornea for the time period to a baseline FOS property. A health property of the cornea can be determined based on the comparison.

In some instances, a system can be used for measuring FOS from one or more nerves in the patient's cornea (e.g., using an OCT-based method). The system can include a mechanism (e.g., an OCT mechanism) configured to perform an optical method on a cornea of a patient for a time period to detect transient optical changes corresponding to Fast Optical Signals (FOS) of the action potentials generated by one or more nerves of the cornea. The system can also include a computing device having at least a memory storing instructions and a processor to execute the instructions to analyze the FOS of the action potentials by: quantifying a property of the FOS of the action potentials generated by the one or more nerves of the cornea for the time period and comparing the property of the FOS of the action potentials generated by the one or more nerves of the cornea for the time period to a baseline FOS. A health property of the cornea of the patient can be determined based on the comparison.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram showing an example of a system that can detect fast optical signals (FOS) of action potential firing from one or more nerves in a patient's cornea to monitor and diagnose the health and wellness of the patient;

FIGS. 2 and 3 are block diagrams showing examples of the optical device of FIG. 1;

FIG. 4 is a process flow diagram of an example of a method for detecting FOS of action potential firing from one or more nerves in the patient's cornea to monitor and diagnose the health and wellness of the patient;

FIGS. 5 and 6 are process flow diagrams of example of methods for identifying changes in a signal response of a corneal nerve corresponding to FOS;

FIG. 7 is a process flow diagram of an example of a method for determining an effectiveness of a treatment for a condition related to one or more corneal nerves; and

FIG. 8 is a process flow diagram of an example of a method for determining whether a medical intervention for a condition related to one or more corneal nerves is necessary.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising.” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the terms “optical method” and “optical technique” refer to any type of analysis that uses light to probe or control matter. The optical method may include an imaging modality, like optical coherence tomography, and may also include additional (non-imaging) steps (like neural stimulation/activation).

As used herein, the term “optical coherence tomography”, also referred to as “OCT”, refers to an optical technique that uses low-coherence interferometry and backscatter contrast to image tissue and other scattering samples and is clinically used and accepted. In an example, OCT can be thought of as analogous to ultrasound imaging, except that OCT uses light instead of sound to acquire sub-surface images, such that terms like “optical ultrasound” are often used to describe OCT.

As used herein, the term “fast optical signals”, also referred to as “FOS”, refers to transient optical changes that occur in neural tissue when the neural tissue is active (depolarized or hyperpolarized). Neural tissue can be active before, during, or after the firing of one or more action potentials. FOS can provide high-resolution information regarding nervous tissue that can be measured in a non-contact manner as transient optical changes occur. There may be several mechanisms underlying the transient optical changes behind FOS, but they likely include phase changes from neuron membrane motion (expansion and contraction), refractive index, scattering, birefringence (polarization properties), and other optical changes around the neuron corresponding to ionic fluxes during action potentials.

As used herein, the term “cornea” relates to the largely transparent part of the eye that is densely innervated (with corneal nerves, also referred to interchangeably as nerves of a patient's cornea). For example, the corneal nerves can be responsible for the sensations of touch, pain, and temperature, and provide an important role in the blink reflex, wound healing, tear production, and secretion. Corneal nerve dysfunction is a significant and growing public health problem, which can be affected by age, damage, ocular surgeries, and various diseases that cause peripheral neuropathies, such as diabetes. It should be understood that the cornea can have different nerve types present, and the different nerve types can be assessed by responses to specific types of stimuli, allowing assessment of changes to different nerve populations.

As used herein, the term “health property” of the cornea can describe anything related to corneal nerve dysfunction that can be indicated by one or more occurrences of FOS. For example, health properties can be reflected in nerve problems that can in turn lead to diseases detrimentally affecting millions of people, such as corneal nerve dysfunction, corneal neuropathy, mild neurotrophic keratopathy, dry eye syndrome (DES), one or more neuropathic pain syndromes (such as diabetic neuropathy, neuropathy related to a neuropathic pain syndrome, neuropathy related to radiation therapy, and/or neuropathy related to herpes simplex or herpes zoster infection), or the like. The health property can be linked to one or more other conditions or diseases, such as pain, depression, neuropathies of peripheral nerves and/or the central nervous system, diabetes, overall patient wellness, or the like.

As used herein, the term “patient” refers to an animal, such as a human.

### II. Overview

The structure and function of corneal nerves can reveal much about the patient's health and wellness. In fact, corneal nerve dysfunction is a significant and growing public health problem, with nerve health and normal function being detrimentally affected by factors including age, damage, ocular surgeries, and various diseases that cause peripheral neuropathies, such as diabetes. Indeed, corneal nerve related problems, such as dry eye syndrome and neuropathic pain, can detrimentally affect millions of people. Traditional methods for assessing the structure and function of a patient's corneal nerves cannot precisely and accurately study the activity of a patient's corneal nerves. Additionally, traditional methods of studying corneal nerve activity cannot be used for diagnostics until significant, and often irreversible, damage has already occurred.

Fast optical signals (FOS) have been shown to accompany neural activity and recently it has been shown that FOS can be detected in thin tissues using optical methods without contrast agents. The cornea is relatively thin, largely transparent, and highly innervated, which makes corneal nerves an ideal candidate for optical methods of FOS detection that do not use contrast agents. Accordingly, optical changes indicative of FOS accompanying conduction of one or more corneal nerves can be detected using optical methods (e.g., using optical coherence tomography (OCT) or the like) to assess corneal nerve functioning and correlated to the patient's health and wellness. Optical methods like OCT are already clinically used and accepted for optical imaging of structures in the eye. Improved methods of optical imaging, such as using traditional OCT with improved analytics and imaging targets, can allow for high resolution, non-contact neural signaling measurements of individual neurons in humans.

### III. Systems

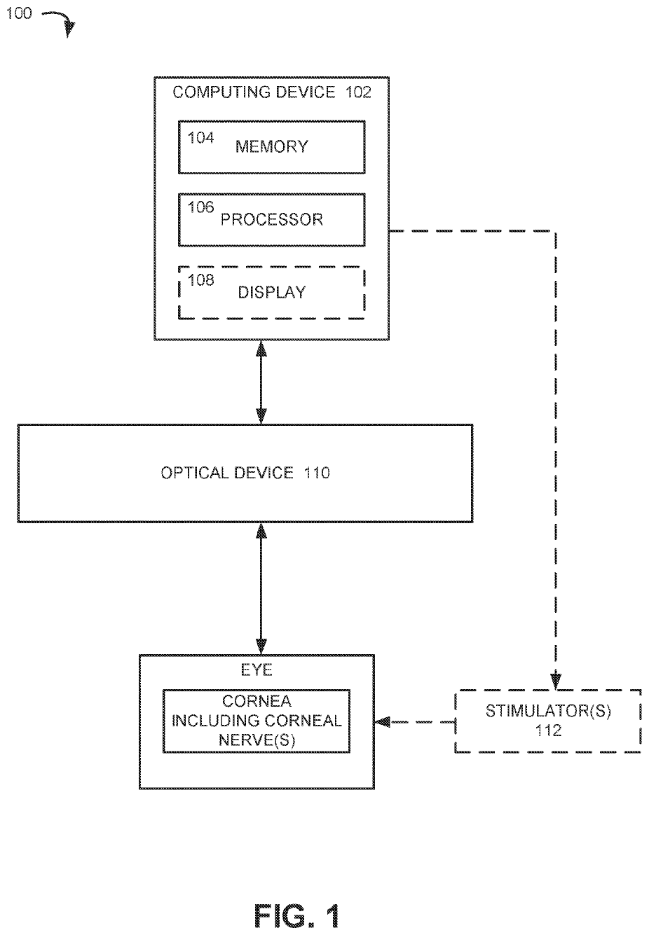

An aspect of the present disclosure can include a system 100 (FIG. 1) that uses optical methods (e.g., like optical coherence tomography (OCT)) to measure fast optical signals (FOSs) from one or more corneal nerves for monitoring the patient's health and wellness. The system 100 can include a computing device 102 and an optical device 110 (also referred to as “an optical mechanism”). The computing device 102 and the optical device 104 can be within a single device or group of connected devices or can be entirely separate devices. In some instances, the system 100 can include at least a portion of devices that are already used in an optometry setting with adjustments (e.g., to software run by/on the computing device 102) to accommodate the measurement of FOS and the associated analysis/output. In some instances, the system 100 can also include one or more stimulator(s) 112.

The optical device 110 can be a device that is traditionally used in optometry for other purposes (e.g., an OCT device). The optical device 110 can be configured to perform an optical method on a cornea (for a time period dictated by software). The optical device 110 can be an imaging device and/or a viewing device that does not record images. Based on the optical method, transient optical changes corresponding to FOSs of the action potentials generated by one or more nerves of the cornea can be detected (e.g., by a detection mechanism that may be part of the optical device 110) and reported to the computing device 102.

The computing device 102 can include at least a memory 104 storing instructions/data and a processor 106 to execute the instructions. The memory 104 can be a non-transitory storage device (e.g., RAM, ROM, solid state device, etc.). The processor 106 can be a hardware device configured to execute instructions stored in the memory 104. In some instances, the processor 106 and memory 104 can be part of the same device (e.g., a microprocessor). In other instances, the processor 106 and memory 104 can be different devices. In some instances, the computing device 102 can have additional components, like input and output components (e.g., display 108 that may be audio and/or visual, a user interface such as a keyboard, mouse, touch screen, or the like). The computing device 102 can command the optical device 110 based on the instructions and can receive data from the optical device.

Upon receiving the data related to the transient optical changes corresponding to FOSs of the action potentials generated by one or more nerves of the cornea from the optical device 110, the computing device 102 can analyze the FOSs of the action potentials. The analysis can include, but is not limited to, quantifying a property of the FOSs of the action potentials generated by the one or more nerves of the cornea for the time period; and comparing the property of the FOSs of the action potentials generated by the one or more nerves of the cornea for the time period to a baseline FOS. The baseline FOS can be based on data from a population of people, data from a population of people with a similar condition, and/or from historical data from the patient. The property of the FOSs of the action potential can be, for example, a temporal pattern of FOS, a number of FOS events, a frequency of FOS, an amplitude of the FOS, any measurement of the waveform of the FOS. A health property of the cornea of the patient may be determined based on the comparison.

Clinically, an FOS-based neural function measurement can be used in several ways. Primarily, the FOS-based neural function measurement can allow for assessment of nerve health based on unperturbed basal signaling with or without blinking, in response to tear film breakup, or the response of the nerves (as measured by FOS) to various types of stimulation (e.g., mechanical, chemical, thermal, or the like). For instance, traditionally, physicians presented with patients reporting dry eye disease often struggle to identify the root cause of the disorder and will attempt iterative treatment plans addressing different potential aspects of the disease. In order to better determine if the root cause is neuropathic (and thus indicate a neuropathic-focused treatment) physicians can use the system 100 to directly assess the functionality of the ocular surface nerves, either in a natural state (such as over the course of a blink reflex) or as a structured sensitivity exam using stimuli. In a structured exam, responses would be assessed by quantifying the property of FOSs measured after a given stimulation (e.g., mechanical, temporal, chemical, or the like given by stimulator(s) 112) and comparing that property to a healthy range of what FOS would be expected for that stimulation (determined by a study of healthy patients, or previous measurements of the patient when healthy). The property of FOS can be, but is not limited to, the temporal pattern of FOS, number of FOS events, frequency of FOS, the amplitude of the FOS, any measurement of the waveform of the FOS, or the like. The property can be based on a stimulated response or a natural (non-stimulated) response.

This FOS-based neural function measurement can be done in a variety of ways, such as comparing the FOS measured responses per unit surface area of the cornea. The spatial resolution of such a measurement can also be used to assess nerve damage and healing following ocular surgeries, which can often involve severing some of the corneal nerves. A longitudinal series of measurements of a patient's individual FOS responses to stimulation over time can be used to assess the progression of neuropathies or direct therapies and treatments. Comparisons can be made with previous measurements of the patient's individual FOS responses to determine if a neuropathy has progressed or if a therapy and/or treatment has been at least partially successful. FOS-based measurements can be combined with other diagnostic ocular system measurements, such as measurements of tear film consistency, amount, break up, or the like, in order to diagnose, study, or treat other related biological systems or diseases with more than one symptom, an example of this type of disease is dry eye disease. FOS-based measurements can also be related to patient-reported quality of life surveys and other psychometrics to acknowledge and explore links between corneal neuropathy, pain scales, depression, and other more global measures of patient wellness.

The computing device 102 can include software (e.g., instructions stored in memory 104 and executed by processor 106) that can aid in the diagnosis of illness, as well as improved ability to develop and target therapies for treating the corneal nerves. In some instances, the software can instruct the optical device 110 to perform the optical method on the cornea of the patient to motion register neuron membrane motion and detect transient optical changes. Based on the motion registered neuron membrane motion and transient optical changes the FOS of the action potentials fired from the one or more nerves of the cornea of the patient can be detected by the optical device 110. In other instances, the optical device can perform the optical method on the cornea of the patient to detect the FOS of the action potentials fired from the one or more nerves of the cornea of the patient based on phase changes from neuron membrane motion, expansion and contraction, refractive index, scattering, birefringence, and/or the like.

In some instances, the system 100 can also include one or more stimulator(s) 112 (also referred to as one or more stimulation mechanism). The stimulation mechanism can provide at least one of a mechanical stimulus, a chemical stimulus, or a thermal stimulation. The stimulus can be applied to at least a portion of the cornea of the patient to trigger action potentials to be generated by the one or more nerves of the cornea of the patient. As an example, the stimulation mechanism can be an aesthesiometer and/or a tonometer, each of which can generate a mechanical stimulus (e.g., a force from one or more filaments, a pulse of air, or the like). In some instances, the stimulation mechanism may also be an aesthesiometer capable of stimulating thermal changes. In other instances, the stimulation mechanism may include at least one chemical applied to the cornea of the patient. Examples of stimuli include but are not limited to an air puff to ascertain signaling response to cold receptors, delivery of capsaicin for ascertaining signaling response to pain, carbon dioxide gas puff to ascertain signaling response to different pH, application of an anesthetic to ascertain changings in signaling response, or the like. One or more stimulation mechanisms can be applied to at least a portion of the cornea sequentially and/or in combination in some instances. In other instance, no stimulation mechanism may be applied and naturally triggered action potentials can be assessed. It should be noted that nerve function in the cornea relies on highly redundant nerve arbors and traditional methods can often only measure loss of sensation in the cornea after significant loss of nerve density and function has already occurred. Sub-sensation changes to corneal nerve density and function are important, both for proper trophic function and for maintenance of the corneal epithelium and tear film. Aberrant nerve function can result in diseases such as neurotrophic keratopathy (rare) or dry eye syndrome (common). Therefore, earlier detection of aberrant and/or lost nerve function is of significant importance in improving patient diagnosis, treatments, and outcomes.

Given the dynamic remodeling of nerve structure in the cornea, the response of a population of neurons in the cornea to a predefined stimulus can be assessed by the computing device 102. For example, an aesthesiometer (e.g., stimulator 112) can deliver a known stimulation to at least a portion of a cornea. The aesthesiometer can be a gas aesthesiometer that delivers a puff of gas on the cornea with a known temperature, pH, and/or pressure. The optical device 110 can view and/or image one or more of the nerves of the cornea for a time period that includes at least the time the stimulation is applied. The time period can also include at least one of an amount of time after the stimulation is applied and an amount of time before the stimulation is applied (e.g., half a second before and/or after, 1 second before and/or after, 2 seconds before and/or after, 5 seconds before and/or after, any combination of these amounts of time before and/or after, or the like). The computing device 102 can quantify the response of different neuron types to the known stimulus (e.g., by varying the type of known stimulus and/or a property of the known stimulus) using the view and/or recorded images from the optical device 110. The computing device 102 can also measure sensitivity of the cone or more corneal nerves to stimulus for a given type of neuron by varying the magnitude of the stimulus applied. As another example, responses to the mechanical stimulus, the chemical stimulus, or the thermal stimulation can be averaged to measure signaling of the one or more nerves of the cornea of the patient. The response of the corneal nerves can be compared, by the computing device 102, to a known range of healthy responses to determine if aberrant and/or loss of function has occurred. The response of the one or more corneal nerves can also be compared, by the computing device 102, to one or more previous responses of the one or more corneal nerves of the patient to longitudinally assess the patient's nerve health over time.

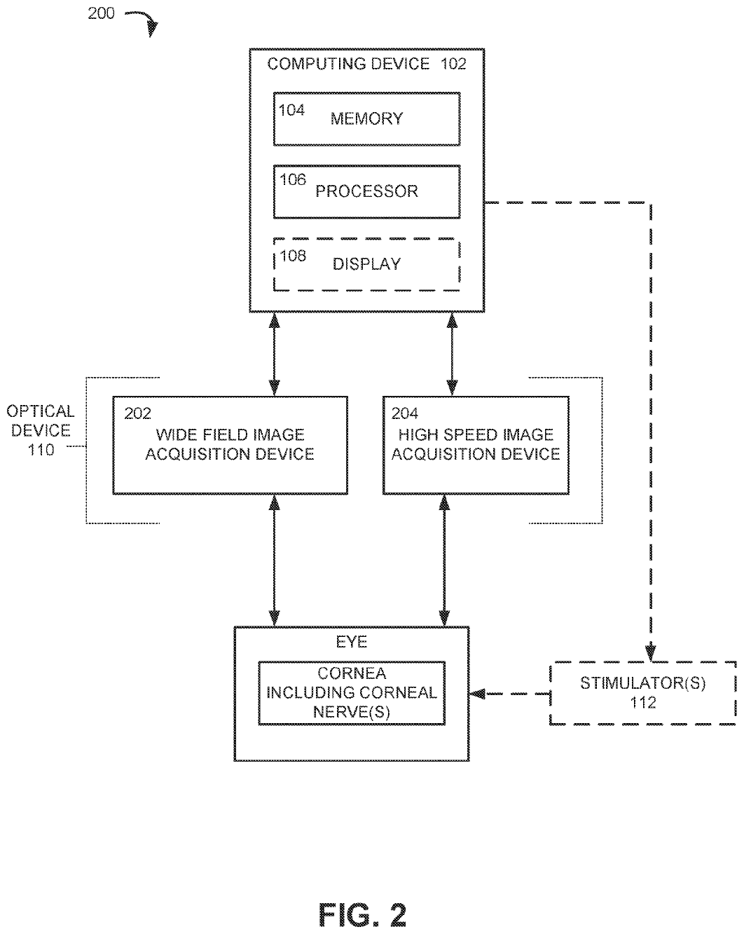

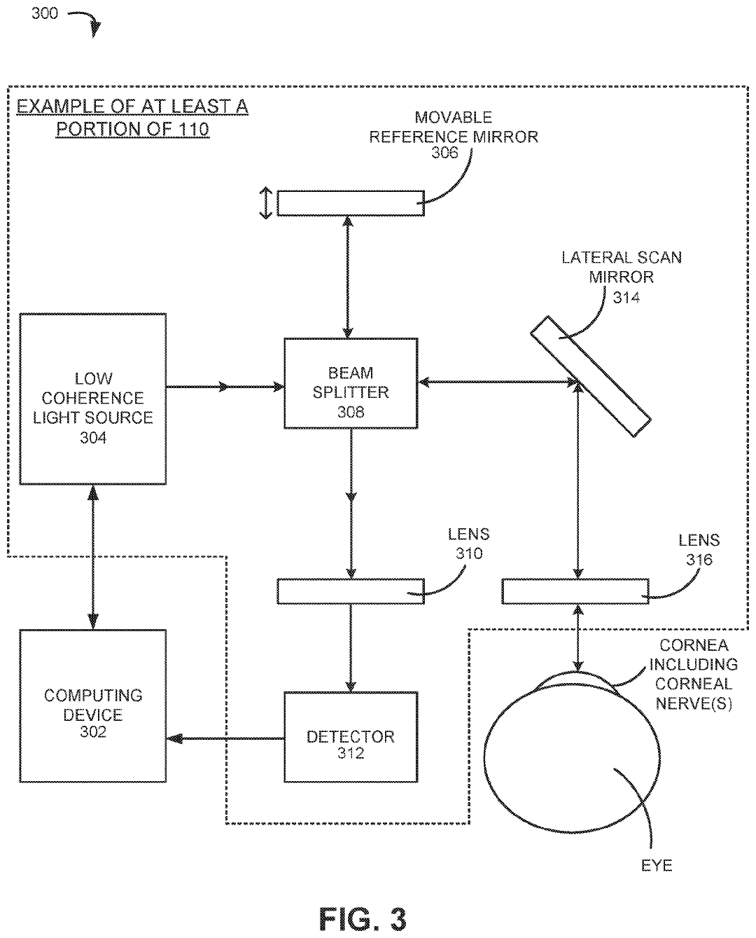

Examples of the optical device 110 are shown in greater detail in FIGS. 2 and 3. In both FIGS. 2 and 3, the optical device 110 can be used to perform OCT. In FIG. 2, the optical device 110 can be two optical devices and/or one device that can be configured for use for two different types of image acquisition. The optical device 110 can include a wide field acquisition device 202 that can be used to acquire a wide field image of at least the cornea of the patient and a high speed image acquisition device 204 that can be used to acquire a zoomed in (compared to the wide field image) image of a portion of the cornea including one or more corneal nerves at a higher focus. The wide field image acquisition device 202 can acquire a wide field image of at least the cornea of the patient, based on instructions from the computing device 102, to identify the location of one or more corneal nerves of interest. Then the high speed image acquisition device 204 can target and image, based on instructions from the computing device 102, a region of interest based on, for example, nerve morphology and/or specific corneal structures. The high speed image acquisition device 204 can image the targeted region during basal (natural) signaling (e.g., no stimulation applied) and/or during intentional stimulation (e.g., using one or more of stimulator(s) 112). The computing device 102 can ascertain the signaling responses and observe and record changes in the signaling responses (the FOSs) of the one or more corneal nerves. The computing device 102 can then compute one or more health properties of the patients based on the changes (or absence of changes) in the signaling responses.

In FIG. 3, the schematic of at least a portion of the optical device 110 is shown communicating with a computing device 302 (which may be the same as computing device 102 but need not be). The schematic shows an example system capable of OCT, but is should be understood that other known system configurations capable of OCT can be used interchangeably. The schematic includes a low coherence light source 304, a beam splitter 308, a moveable reference mirror 306, a lens 310, a detector 312, a lateral scan mirror or mirrors 314, and a lens 316, all of which are required to deliver light to the cornea and/or receive a reflection from the cornea. The low coherence light source 304 can emit light towards the beam splitter 308. The beam splitter 308 can split the light towards a movable reference mirror 306 and a sample (the cornea). The beam splitter 308 can then split the light towards a lateral scan mirror 314 that directs the light through a lens 316 to the cornea including the one or more corneal nerves. The light can be focused at different positions and/or depths of the cornea by moving the movable reference mirror 306 and/or the lateral scan mirror 314 to receive reflections of the cornea (including the one or more corneal nerves). The reflected light can be directed back through lens 316 and to the beam splitter 308 via the lateral scan mirror 314. The beam splitter 308 can recombine the light directed back from the lens 316 and the reference mirror 306. The beam splitter 308 can then direct the reflected light through a lens 310 to the detector 312 which can communicate images based on the reflected light to the computing device 302 (or, not shown, viewable by a medical professional via one or more view ports).

In FIGS. 2 and 3, OCT can be performed by the optical device 110 to detect the FOS of one or more corneal nerves. The OCT can provide a repeatable assessment standard for corneal nerve health and identification of changes in nerve function over time. Functional measurements in the cornea can be accomplished by measuring FOS for a time period using the optical device 110 and computing device 102 and/or 302 to quantify discrete FOS in individual nerve tracts or larger nerve bundles. The computing device 102 and/or 302 can include software that can record, count, analyze, and/or quantify the discrete FOS signals. As an example, the systems 100, 200, and/or 300 (by the computing device 102 and/or 302) can determine the efficacy of therapies, such as, but not limited to, nerve growth factor (NGF) applied for alleviating and/or treating dry eye syndrome and/or neurotrophic keratopathy, and/or track the progression of corneal nerve dysfunction over time. As another example, the systems 100, 200, and/or 300 (by the computing device 102 and/or 302) can define the relationship between nerve function of one or more of the corneal nerves and sensitivity (e.g., as measured by aesthesiometers) and/or establish the relationship between corneal nerve function of the one or more corneal nerves and the function of the Meibomian glands that secrete the tear film, allowing for better clinical assessment and decision making around treatment of dry eye syndrome. FOS-based corneal nerve measurements using OCT can also assess the progression of system peripheral neuropathies that affect more than one portion of the body, such as diabetic related neuropathies, and can be a tool to diagnose and direct treatment of other nerve system problems that are less accessible to diagnostics.

OCT-based measurements of FOS of corneal nerve activity using the systems 100, 200, and/or 300 can use motion registration. Detection of neural activity can be based on optical changes, such as phase shifts. Phase noise in the measurement FOS of corneal nerve activity can be reduced by using the reflective surface of the cornea being measured as a reference to reduce phase noise. The magnitude of FOS of corneal nerve activity detected by the systems 100, 200 and/or 300 can be boosted by temporal averaging of data relative to a stimulation of the cornea (e.g., mechanical, chemical, thermal, etc.). Spatial averaging can also be done along the nerve. Given that OCT is non-contact and already widely used in ophthalmology, and is thus familiar to doctors and patients alike, OCT is an ideal imaging modality for an ophthalmic neural assessment of FOS of corneal nerves.

### IV. Methods

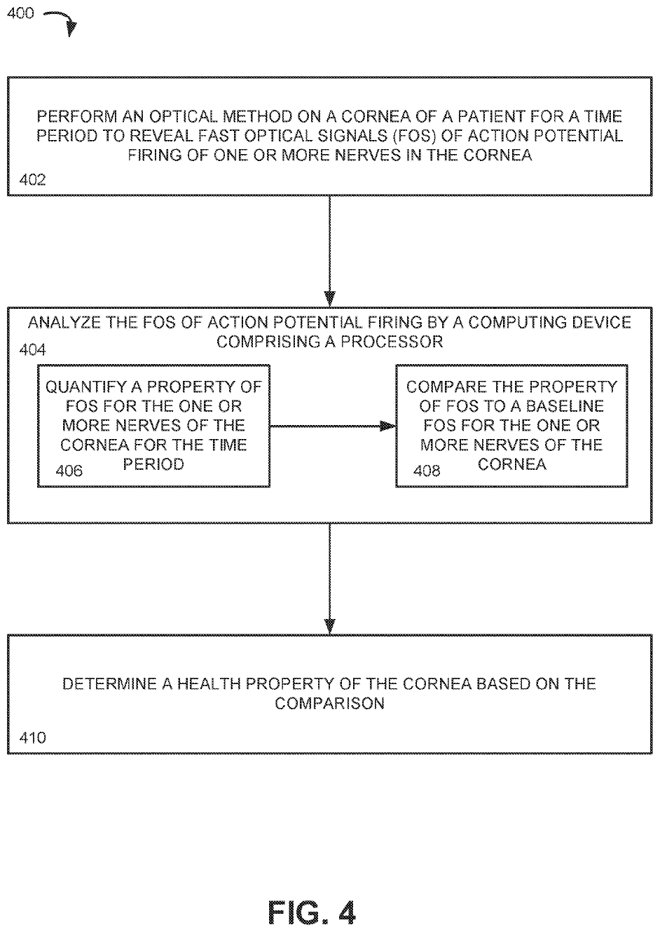

Another aspect of the present disclosure can include methods 400-800 (FIGS. 4-8) that can be used alone or in combination for monitoring a patient's health and wellness. The patient's health and wellness can be monitored using methods 400-800 by detecting and measuring fast optical signals (FOS) from one or more nerves in the patient's cornea using an optical method (e.g., using optical coherence tomography (OCT)). Components of the systems 100-300 can be used for executing the methods 400-800. Certain optical views and/or images, such as OCT images, can show the FOS of one or more one corneal nerves. The FOS of the one or more corneal nerves can be used to assess the function of the one or more corneal nerves. The assessed function of the one or more corneal nerves can be used to determine if the patient has one or more diseases, disorders, or symptoms, such as: corneal nerve dysfunction, corneal neuropathies, mild neurotrophic keratopathy, and neuropathic dry eye syndrome based on the health property. The assessed function of the one or more corneal nerves can also be used to determine if a therapy and/or treatment for one or more diseases, disorders, or symptoms is working over time.

For purposes of simplicity, the methods 400-800 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 400-800, nor are methods 400-800 limited to the illustrated aspects. Different terminology may be used to define terms/components in the systems and methods, but it should be noted that the methods 400-800 describe use of the systems 100-300.

The method 400 shown in FIG. 4 detects the FOS of action potential firing from one or more nerves in the patient's cornea (e.g., corneal nerves) to monitor and diagnose the health and wellness of the patient. At 402, an optical method can be performed on a cornea of a patient to record images of the cornea for a time period to reveal Fast Optical Signals (FOS) of action potential firing of at least one corneal nerve in the cornea. FOS represent transient optical changes corresponding to at least one of ionic fluxes during action potentials, phase changes from neuron membrane motion, expansion and contraction, refractive index, scattering, birefringence, and the like. The optical method can include using optical coherence tomography (OCT) or another imaging or viewing method used commonly in ophthalmology. The optical method can motion register at least one of the transient optical changes described above to reveal FOS. For example. FOS can be expressed by the corneal nerves after depolarization (which may be due to stimulation). FOS can be detected with OCT scanning based on the conduction. In some instances, the optical method can include additional steps, such as stimulating the cornea with a stimulus that is configured to trigger action potentials in the one or more corneal nerve. For example, the cornea can be stimulated (e.g., with an aesthesiometer or a puff of gas) with at least one of mechanical, chemical, or thermal stimulation to trigger action potentials in the at least one corneal nerve. The FOS can be detected via the optical method before and/or during and after the stimulation of the cornea using motion registration and detection of transient optical changes.

At 404, the FOS of action potential firing of the one or more corneal nerves can be analyzed by a computing device comprising a processor (e.g., any device that includes at least processing capability). The analyzing can include quantifying at least one property of the FOS for the one or more corneal nerves for the time period (at 406) and comparing the at least one property of the FOS to a baseline FOS for the one or more corneal nerve (at 408). The baseline FOS can be determined from a population of people, from a population of people with a similar condition, and/or from historical data from the patient. The baseline FOS can data for one or more properties of FOS. The property of FOS can be, but is not limited to, the temporal pattern of FOS, number of FOS events, frequency of FOS, the amplitude of the FOS, any measurement of the waveform of the FOS, any of these characteristics in a response of FOS to stimuli, or the like. It should be noted that the method 400 can be used for one or more nerves, individual nerve tracts, or larger nerve bundles of the corneal nerve. As another example, responses to the mechanical stimulus, the chemical stimulus, or the thermal stimulation can be averaged to measure signaling of the one or more nerves of the cornea of the patient.

At 410, a health property of the cornea and/or the patient overall can be determined (e.g., manually and/or in an automated fashion) based on the comparison between the baseline FOS and the at least one quantified property of the FOS. In some instances, more than one health property can be determined at a time. For example, the health property of the cornea can be linked to pain, depression, neuropathies of peripheral nerves and/or the central nervous system, and/or overall patient wellness. Based on the determined health property (or properties) an efficacy of a therapy affecting the health property (e.g., nerve growth factors applied to the cornea of the patient, anti-inflammatoires, anti-depressants, anticonvulsants, ocular surgery, or the like) can be tracked. Additionally, and or alternatively, the progression of a disease (e.g., diabetic neuropathy, neuropathy related to a neuropathic pain syndrome, neuropathy related to radiation therapy, neuropathy related to herpes simplex or herpes zoster infection, or the like) over time can be tracked or predicted based on the health property (or properties).

Clinically, an FOS-based neural function measurement detected as part of method 400 can be used in several ways. The FOS-based neural function measurements can be used to assess nerve health based on unperturbed basal signaling with or without blinking, in response to tear film breakup, and/or in response to various types of stimulation (e.g., mechanical, chemical, thermal, or the like). For instance, traditionally, physicians presented with patients reporting dry eye disease often struggle to identify the root cause of the disorder and attempt iterative treatment plans addressing different potential aspects of the disease (e.g., a guess and check or elimination plan). In order to better determine if the root cause is neuropathic (and thus indicate a neuropathic-focused treatment) physicians can directly assess the functionality of the ocular surface nerves, either in a natural state (such as over the course of a blink reflex) or as a structured sensitivity exam using stimuli using the methods described herein. In a structured exam, responses can be assessed by quantifying at least one property of FOSs measured after a given stimulation (e.g., mechanical, temporal, chemical, or the like) and comparing that at least one property to a healthy range of what FOS would be expected for that stimulation (e.g., determined by a study of healthy patients and/or previous measurements of the patient when healthy). The at least one property of FOS can be, but is not limited to, at least one of the temporal pattern of FOS, number of FOS events, frequency of FOS, the amplitude of the FOS, any measurement of the waveform of the FOS, or the like. The property can be based on a stimulated response or a natural (non-stimulated) response.

This FOS-based neural function measurement can be done in a variety of ways, such as by comparing the FOS measured responses per unit surface area of the cornea. The spatial resolution of such a measurement can also be used to assess nerve damage and healing following ocular surgeries, which can often involve severing some of the corneal nerves. A longitudinal series of measurements of a patient's individual FOS responses to stimulation over time can be used to assess the progression of neuropathies or direct therapies and treatments. Comparisons can be made with previous measurements of the patient's individual FOS responses to determine if a neuropathy has progressed or if a therapy and/or treatment has been at least partially successful. FOS-based measurements can be combined with other diagnostic ocular system measurements, such as measurements of tear film consistency, amount, or the like, in order to diagnose, study, or treat other related biological systems or diseases with more than one symptom, such as in dry eye disease. FOS-based measurements can also be related to patient-reported quality of life surveys and other psychometrics to acknowledge and explore links between corneal neuropathy, pain scales, depression, and other more global measures of patient wellness.

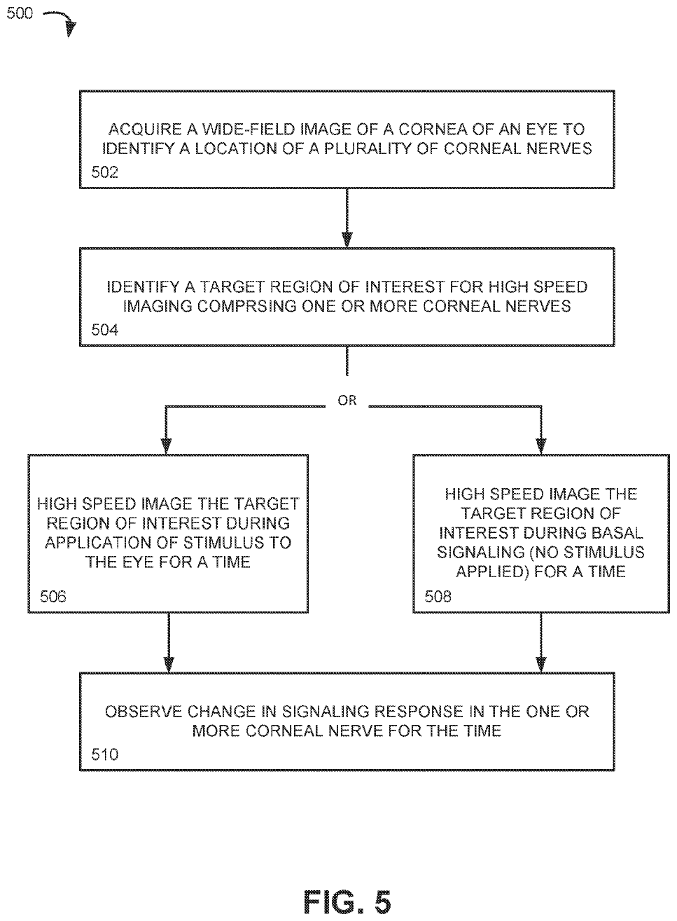

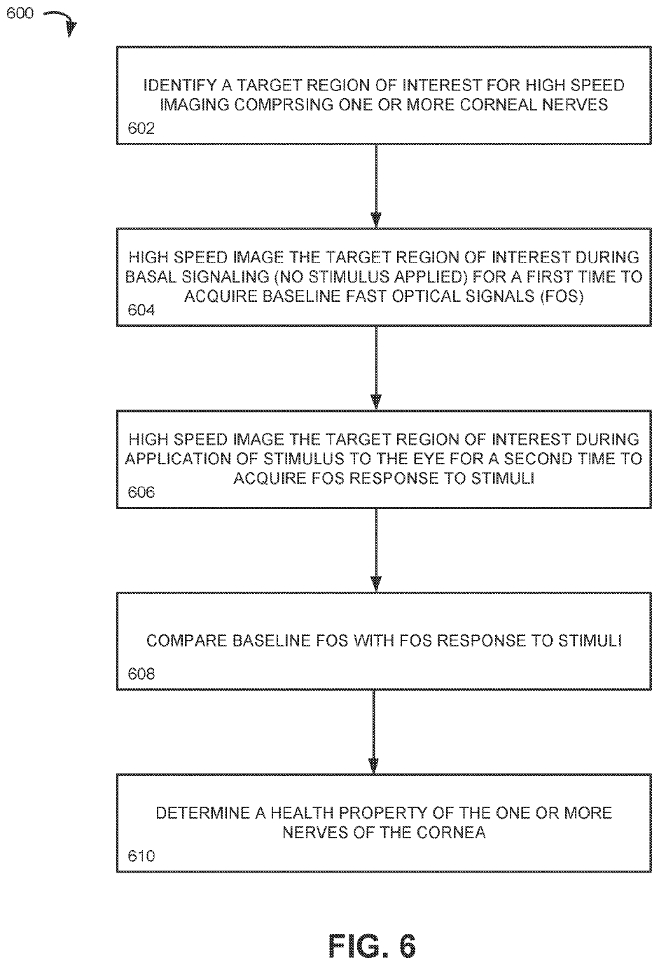

FIGS. 5 and 6 show example methods 500 and 600, respectively, for identifying changes in a signal response of one or more corneal nerves by detecting FOS of the one or more corneal nerves. Both or either of FIGS. 5 and 6 can be used to execute at least one of the steps of the method 400 of FIG. 4 (e.g., performing an optical method on the cornea to reveal FOS).

Instrumentation traditionally utilized in an optometrist's office (e.g., OCT) can be utilized to conduct the method 500 and/or 600. OCT of FOS of one or more corneal nerves can provide a repeatable assessment standard for corneal nerve health and identification of changes in nerve function over time. Functional measurements in the cornea can be taken to quantify discrete FOS in individual nerve tracts or larger nerve bundles. Software can be used to record, count, analyze, and/or quantify the discrete FOS signals. OCT-based measurements of FOS of corneal nerve activity can use motion registration and detection of neural activity based on optical changes, such as phase shifts. Phase noise in the measurement FOS of corneal nerve activity can be reduced by using the reflective surface of the cornea being measured as a reference to greatly reduce phase noise. The magnitude of FOS of corneal nerve activity detected can be boosted by temporal averaging of data relative to a stimulation of the cornea (e.g., mechanical, chemical, thermal, etc.). Spatial averaging can also be done along the nerve. Given that OCT is non-contact and already widely used in ophthalmology, and is thus familiar to doctors and patients alike, OCT is an ideal imaging modality for an ophthalmic neural assessment of FOS of corneal nerves.

As shown in FIG. 5, the method 500 begins at 502. At 502, a wide-field image of the cornea of a patient's eye can be acquired to identify the location of a plurality of corneal nerves within the cornea. At 504, a target region of interest for high speed imaging can be identified, based on at least eh wide-field image. The target region of interest can include one or more corneal nerves. At 506, the target region of interest can be high speed imaged (e.g., via OCT) during application (and after application) of a stimulus (e.g., mechanical, chemical, thermal, or the like) to the eye for a time to detect FOS of the one or more corneal nerves in response to the stimulus. Alternatively, at 508, a stimulus is not applied to the eye and a high speed image of the target region of interest is taken during basal signaling of the one or more corneal nerves for a time. Thus, a natural FOS can be detected from the one or more corneal nerves. One or more FOS can be detected for each of the one or more corneal nerves. After both 506 and 508, at 510, changes in the signaling response in the one or more corneal nerves for the time can be observed based on the detected FOS. The changes can be observed based on comparisons with baseline signaling responses for the patient or a population of similar patients.

As shown in FIG. 6, the method 600 begins at 602. At 602, a target region of interest for high speed imaging comprising one or more corneal nerves can be identified. The target region of interest for high speed imaging can be identified based on a wide field image acquired as described above with respect to method 500 of FIG. 5. At 604, the high speed image of the target region of interest can be taken during basal signaling with no stimulus applied for a first time to acquire a baseline FOS (e.g., a baseline signaling response based on the detected FOS and/or at least one quantifiable property of the detected FOS). It should be understood, however, that the baseline can be determined (additionally or alternatively) from a population of people, from a population of people with a similar condition, from historical data from the patient, and the like. At 606, a high speed image can be taken of the target region of interest during (or after, while the effects are still being felt) application of a stimulus to the eye at a second time to detect an FOS (and/or at least one quantifiable property of the FOS) indicative of a signaling response in response to the one or more stimuli. For example, the stimulus can be a mechanical stimulus (provided by an aesthesiometer, a tonometer, or the like), a chemical stimulus, a thermal stimulus, or the like, designed to trigger one or more action potentials of one or more corneal nerves. Examples of stimuli include but are not limited to a menthol gas puff to ascertain signaling response to cold receptors, delivery of capsaicin for ascertaining signaling response to pain, carbon dioxide gas puff to ascertain signaling response to different pH, application of an anesthetic to ascertain changings in signaling response, or the like.

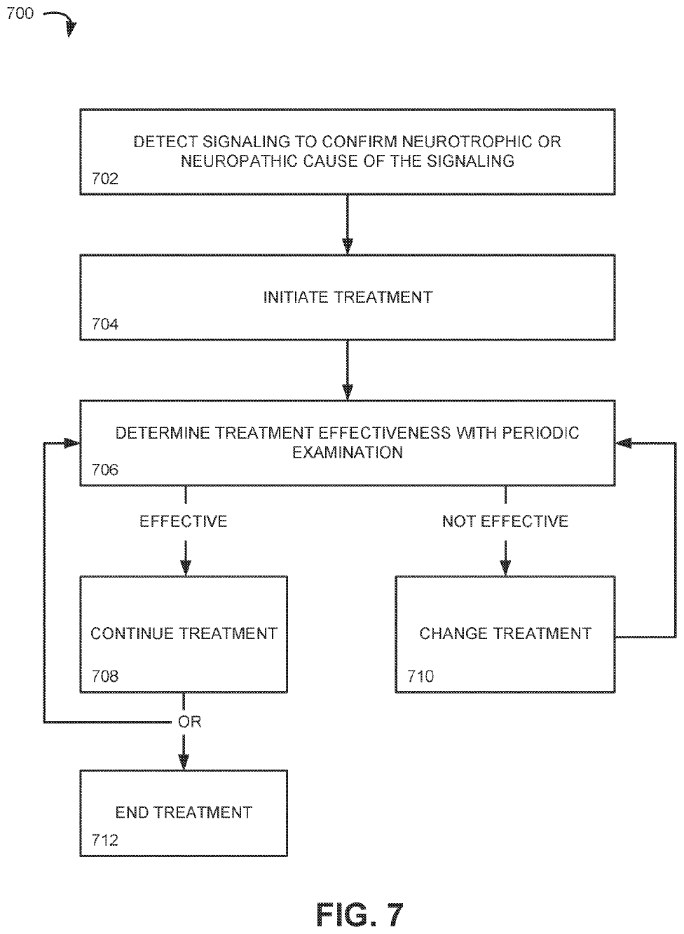

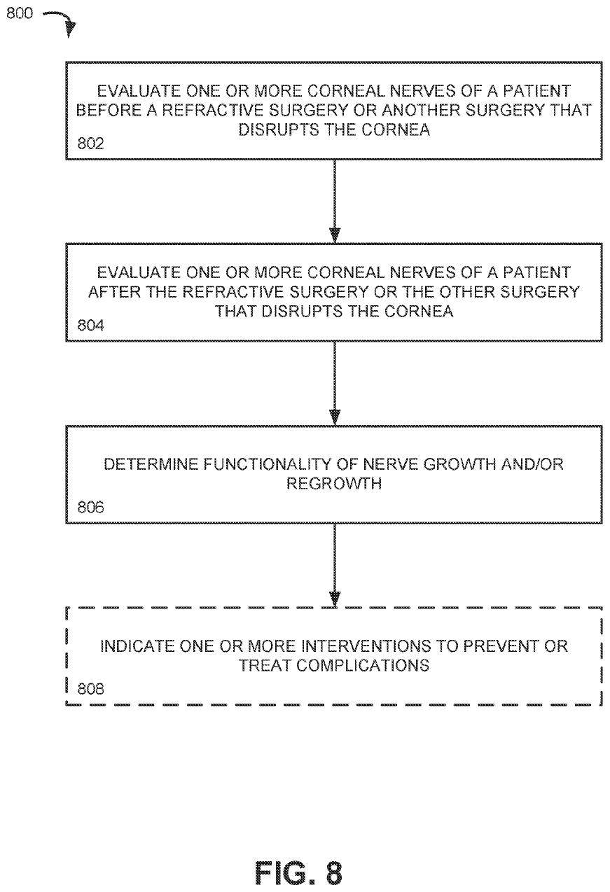

At 608, the baseline FOS can be compared to the FOS response to stimuli and any difference can be determined. For example, there can be a difference in one or more quantifiable property of the FOS. Based on the difference, a health property of the one or more nerves of the cornea or the patient as a whole can be determined. The health property can be correlated to one or more diseases or conditions, such as has one or more of: corneal nerve dysfunction, corneal neuropathies, mild neurotrophic keratopathy, neuropathic dry eye syndrome, or the like. Indeed, the progress of the disease can be tracked over time using multiple evaluations of signaling responses of the corneal nerves based on the health property. An efficacy of a therapy (e.g., nerve growth factors, anti-inflammatories, anti-depressants, anticonvulsants, ocular surgery, or the like) affecting the health property can also be tracked, or the like. Other example uses of tracking of corneal nerve signaling responses detected via FOS are shown in FIGS. 7 and 8.

Referring now to FIG. 7, illustrated is a method 700 for determining an effectiveness of a treatment or therapy for a condition related to one or more corneal nerves. At 702, signaling can be detected to confirm a neurotrophic or neuropathic cause of the signaling. The signaling can be detected using OCT to image FOS from one or more corneal nerves. The detection can include detecting if the signaling is normal or aberrant and/or detecting what nerve/receptor type are affected. At 704, a treatment can be initiated if a neurotrophic or neuropathic cause was detected. For example, a nerve growth factor treatment can be initiated. At 706, an effectiveness of the treatment can be determined with periodic examination of the FOS of the one or more corneal nerves using the optical methods described above. When the treatment is deemed to be effective, at 708 the treatment can be continued or if the condition is satisfactorily treated, the treatment can be ended. If the treatment is not effective, at 708, the treatment can be changed/altered and applied and then evaluated over time similar to the previous treatment.

Referring now to FIG. 8, illustrated is a process flow diagram of a method 800 for determining whether a medical intervention for a condition related to one or more corneal nerves is necessary. At 802, one or more corneal nerves of a patient can be evaluated before a refractive surgery or any other surgery that disrupts the cornea. The one or more corneal nerves can be evaluated by performing an optical method to detect the FOS of the one or more corneal nerves and determining the functioning of the one or more corneal nerves (e.g., based on at least one quantified property of the FOS) prior to any procedure. The surgery can be performed, and then at 804, the one or more corneal nerves can be evaluated after the refractive surgery or the other surgery that disrupts the cornea. The one or more corneal nerves can be evaluated by performing an optical method to detect the FOS of the one or more corneal nerves and determining the functioning of the one or more corneal nerves (e.g., based on at least one quantified property of the FOS). The one or more corneal nerves can be evaluated multiple times after a surgery (e.g., on the day of the surgery, a day after the surgery, a week after the surgery, a month after the surgery, six months after the surgery, or the like) to determine healing progression, success of the surgery, failure of the surgery, complications, or the like. At 806, the functionality of nerve growth and/or regrowth can be determined, for example based on a comparison of the before and after surgery evaluations and/or based on a comparison of one or more previous after surgery evaluations with a most recent evaluation. Optionally, at 808, one or more interventions can be indicated that prevent or treat complications that may have been noticed during 806.

As an example, a patient may experience ocular pain despite no obvious signs of ocular surface disease, as determined by traditional assessment methods such as fluorescein break-up time, Schirmer tear test, or the like. The technology described herein can detect for aberrant signaling in one or more corneal nerves and if aberrant signaling is detected based on the FOS, then a neurotrophic or neuropathic cause can be confirmed, where before no cause could be determined. A treatment for such underlying cause, such as appropriate application of neural growth factor can then be indicated and applied. Once a treatment has been initiated, the patient can be periodically examined, using the optical methods described above, in the same region of interest to determine if aberrant or overacting signaling is being ameliorated by the treatment. It should be noted that neural growth factor treatments are presently very expensive, so precise diagnosis and monitoring are required. In another example, a patient who received refractive surgery (such as LASIK or other corneal ablative or incisional procedures), or other surgery that can disrupt or otherwise affect the cornea (such as cataract surgery), can be evaluated before and/or after the procedure. The evaluation can be over the entirety of the cornea or at an incision site in order to determine functionality of nerve (re) growth and to indicate if early interventions are necessary to prevent or stop the spread of complications (such as dry-eye secondary to surgery or corneal scarring due to persistent epithelial defects). In a further example, the FOS responses of specific receptor/nerve types in an individual patient (or for a population in general given enough individual patient data) can be mapped. The mapping can be done by synthesizing information from evaluations of FOS when varied stimuli (e.g., menthol, capsaicin, CO2, anesthetic, or the like) are applied to at least a portion of the cornea. Types of aberrant signaling can be localized to a specific receptor/nerve type. Based on the mapping, therapies can be targeted at the specific receptor/nerve type and treatment effects can be monitored as described above to improve understanding of corneal nerve related diseases, disorders, and symptoms and to better treat patients.

From the above description, those skilled in the art will perceive improvements, changes, and modifications. Such improvements, changes and modifications are within the skill of one in the art and are intended to be covered by the appended claims.

The following is claimed:

1. A method comprising:
performing an optical method on a cornea of a patient for a time period to detect transient optical changes in one or more nerves of the cornea corresponding to Fast Optical Signals (FOS) provided with generation of action potentials by the one or more nerves of the cornea;
receiving, by a computing device comprising a processor, a record of the FOS of the action potentials of the one or more nerves of the cornea and analyzing the FOS of the action potentials generated by the one or more nerves of the cornea, the analyzing comprising:
quantifying a property of the FOS of the action potentials generated by the one or more nerves of the cornea for the time period; and
comparing the property of the FOS of the action potentials generated by the one or more nerves of the cornea for the time period to a baseline FOS property,
wherein a health property of the cornea is determined based on the comparison.

2. The method of claim 1, further comprising stimulating the cornea of a patient with a stimulus that is configured to trigger one or more nerves of the cornea to generate the action potentials.

3. The method of claim 2, wherein the stimulus is at least one of a mechanical stimulus, a chemical stimulus, or thermal stimulus.

4. The method of claim 2, wherein the stimulus is a mechanical stimulus provided by at least one of an aesthesiometer or a tonometer.

5. The method of claim 1, wherein the transient optical changes correspond to phase shifts from neuron membrane motion, expansion and contraction, refractive index, scattering, and/or birefringence.

6. The method of claim 1, further comprising:
determining whether the patient has corneal nerve dysfunction, corneal neuropathies, mild neurotrophic keratopathy, and/or neuropathic dry eye syndrome based on the health property.

7. The method of claim 1, further comprising tracking progress of a disease over time based on the health property.

8. The method of claim 7, wherein the disease is diabetic neuropathy, neuropathy related to a neuropathic pain syndrome, neuropathy related to radiation therapy, and/or neuropathy related to herpes simplex or herpes zoster infection.

9. The method of claim 1, further comprising tracking an efficacy of a therapy affecting the health property, wherein the therapy is at least one of nerve growth factors applied to the cornea of the patient, anti-inflammatories, anti-depressants, anticonvulsants, and/or ocular surgery.

10. The method of claim 1, wherein the baseline FOS is determined from a population of people, from a population of people with a similar condition, and/or from historical data from the patient.

11. The method of claim 1, wherein the one or more nerves comprise an individual nerve tract or a larger nerve bundle.

12. The method of claim 1, wherein the health property of the cornea is linked to pain, depression, neuropathies of peripheral nerves and/or the central nervous system, and/or overall patient wellness.

13. The method of claim 1, wherein optical method comprises optical coherence tomography (OCT).

14. A system comprising:
a mechanism configured to perform an optical method on a cornea of a patient for a time period to detect transient optical changes corresponding to Fast Optical Signals (FOS) of the action potentials generated by one or more nerves of the cornea;
a computing device comprising a memory storing instructions and a processor to execute the instructions to analyze the FOS of the action potentials by:
quantifying a property of the FOS of the action potentials generated by the one or more nerves of the cornea for the time period; and
comparing the property of the FOS of the action potentials generated by the one or more nerves of the cornea for the time period to a baseline FOS,
wherein a health property of the cornea of the patient is determined based on the comparison.

15. The system of claim 14, further comprising a stimulation mechanism configured to apply a mechanical stimulus, a chemical stimulus, or a thermal stimulation to at least a portion of the cornea of the patient to trigger action potentials to be generated by the one or more nerves of the cornea of the patient.

16. The system of claim 15, wherein the processor averages responses to the mechanical stimulus, the chemical stimulus, or the thermal stimulation to measure signaling of the one or more nerves of the cornea of the patient.

17. The system of claim 14, wherein the mechanism configured to perform the optical method is configured for optical coherence tomography (OCT).

18. The system of claim 14, wherein the baseline FOS is based on data from a population of people, data from a population of people with a similar condition, and/or from historical data from the patient.

19. The system of claim 14, wherein the mechanism configured to perform the optical method on the cornea of the patient is configured to motion register neuron membrane motion and detect transient optical changes to detect the FOS of the action potentials fired from the one or more nerves of the cornea of the patient.

20. The system of claim 14, wherein the mechanism configured to perform the optical method on the cornea of the patient is configured to detect the FOS of the action potentials fired from the one or more nerves of the cornea of the patient based on at least one of phase changes from neuron membrane motion, expansion and contraction, refractive index, scattering, and/or birefringence.
